(19) United States
(12) Patent Application Publication
(10) Pub. No.: US 2025/0367358 A1
(43) Pub. Date: Dec. 4, 2025

(54) MALE URINARY COLLECTION DEVICE AND METHOD OF USE

(71) Applicant: Boehringer Technologies, LP, Phoenixville, PA (US)

(72) Inventors: Trevor SMITH, Phoenixville, PA (US); William Charles Dackis, Philadelphia, PA (US); Christopher L. Radl, Malvern, PA (US)

(21) Appl. No.: 19/080,210

(22) Filed: Mar. 14, 2025

Related U.S. Application Data
(60) Provisional application No. 63/653,512, filed on May 30, 2024.

(51) Int. Cl. A61M 1/00

(52) U.S. Cl. CPC A61M 1/74; A61M 1/62; A61M 1/79; A61M 2202/0014; A61M 2202/0496

(57) ABSTRACT

A device and method for removing by suction urine voided by a patient. The device includes an impermealble bag, a wicking pad, a spacer, and an adhesive patch. The bag includes an internal chamber and an entryway through which the penis of the patient is inserted. The wicking pad has a first portion located within the chamber and a second portion located outside the bag in communication with the ambient atmosphere. The adhesive patch secures the device to the patient. The bag includes a port at which regulated suction is provided to carry urine from the patient through the first portion of the wicking pad and out the port. Ambient air flows into the second portion to facilitate the flow of urine therethrough and to aid in drying the wicking pad. The spacer is located outside the bag for enhancing air flow through the second portion.

### CROSS-REFERENCE TO RELATED APPLICATIONS

This utility application claims the benefit under 35 U.S.C. § 119 (e) of Provisional Application Ser. No. 63/653,512, filed on May 30, 2024, entitled “MALE URINARY COLLECTION DEVICE AND METHOD OF USE”, the entire disclosure of that provisional application is incorporated by reference herein for all purposes.

### FIELD OF THE INVENTION

This invention relates generally to medical devices and methods and more particularly to devices and methods for collecting urine from a male patient using suction.

### BACKGROUND OF THE INVENTION

Catheter associated urinary tract infections (CAUTIs), are a common problem in hospitals and result from use of traditional indwelling catheters. Such indwelling catheter devices are placed through a patient's urethra into their bladder where they remain for extended periods of time, in rare cases longer than a week. While the catheter is in place, it becomes a highway for infection. Beyond being a persistent problem for healthcare workers, alert patients are unhappy with the sensation of catheter insertion, removal, or prolonged placement.

CAUTIs not only have potential to cause harm to patients but can also negatively impact a hospital's finances. For example, Medicare severely punishes hospitals when minimum CAUTI allowances are exceeded. Due to the potential for patient harm, resource intensive treatment methods, and potential revenue loss for hospitals, there is a need to reduce the incidence of CAUTIs. One solution has been to remove catheters from the equation entirely and externalize the urine management process. For example, Boehringer Laboratories, LLC, the assignee of the subject invention provides an External Urinary Management System under the trademark CareDry®. That device replaces the classical indwelling catheter with a system including an external sponge connected to a suction source to collect urine from female patients into a collection component as they void before spillage can occur. With that system healthcare workers enjoy easier device placement, patients experience greater comfort, and hospital CAUTI rates decrease due to the elimination of infection facilitating indwelling catheters. Other external catheter devices or systems for female patients are commercially available, such as the Stryker PrimaFit™ device and the Becton Dickinson PureWick™ device

Insofar as male patients are concerned Becton Dickinson offers a male PureWick™ device, and Stryker offers the PrimoFit™ device. Both of those devices are impermable bag constructs which secure around the penis and use suction to collect urine. The patent literature discloses various impermeable bag based devices or systems for collecting urine from a male patient without use of an indwelling catheter. See for example, U.S. Pat. No. 10,376,407 (Newton); U.S. Pat. No. 11,000,401 (Ecklund et al.); and U.S. Pat. No. 11,207,206 (Sharma et al.).

While the above identified commercial and patented urine collecting devices for use by males may be generally suitable for their intended purposes, they nevertheless exhibit one or more of the following problems or are lacking clinically relevant features. These problems or lack of clinically relevant features include: visualization of the male anatomy; moisture retention/drying; leaking or overflow due to patient movement; improper venting; and adhesive self-sticking.

Insofar as visualization of the male anatomy is concerned, all currently marketed products are generally composed of an impermeable bag connected to suction and containing a proprietary combination of layered materials that make up an inside component, referred to hereinafter as the insert material. Generally, the penis is placed through an opening into the bag and sits either in front of or behind the insert material. When the penis is behind the material it cannot be easily seen by healthcare workers to confirm proper placement. If the patient shifts or otherwise moves relative to the device, it can prevent urine from being adequately captured and cause a leak. A primary failure mode of these devices is leaking-failure to capture urine will, at best, necessitate additional patient bathing and bedding changeover; at worst, skin breakdown, infection, and increased hospital stay.

Insofar as moisture retention/drying is concerned, skin breakdown can occur when skin or sensitive organs such as the penis remain in prolonged contact with urine. In devices where the penis rests directly on an impermeable bottom layer of the bag, urine will collect under the penis and remain trapped until the device is replaced with a new one. In devices where the penis rests on the insert material, it will remain in direct contact with urine saturated insert material until that material dries. Drying can take several hours depending on the liquid retention/absorbency of the material layers, airflow through the bag, and the suction setpoint (i.e., the level of suction to be provided to remove the urine from the bag). The properties of the material layers composing the insert material vary among currently marketed devices but generally contain an external permeable membrane housing a series of absorbing or wicking materials. Unfortunately, currently marketed devices absorb too well-once they are saturated with urine, drying is not timely.

Insofar as leaking or overflow due to patient movement is concerned, devices such as the Stryke PrimoFit™ device have an open top design. This allows for high airflow into the device, to prevent negative pressure buildup and injury to the penis. Unfortunately, this open bag design may not be conducive to complete urine removal when the patient is positioned on their side. In healthcare settings bed-bound patients are rotated every couple of hours between their left side, right side, and supine positions-a necessary process to prevent bed sores. When used on side positioned patients, open top devices can be overwhelmed with minimal urine accumulation, traveling up the side of the bag and onto the patient and the bedding. Recently, the Stryke PrimoFit™ device has been replaced by the new PrimoFit+™ device model. The updated model has a larger seal that creates a smaller top opening. This reduces the chance of leaks, but can still be overwhelmed with higher urine void volumes.

Insofar as improper venting is concerned to prevent pressure injury to the penis, external urine collection devices must prevent the internal bag pressure (where the anatomy is placed) from reaching the source/wall pressure. Generally, this is accomplished with openings or vents that allow for the passage of room (ambient) air into the device so that the constant inflow of air keeps the internal device pressure at a safe level. Currently marketed devices are capable of reducing internal pressures relative to a vacuum source but are unable to accommodate certain body shapes. Some of those device struggle with larger patients. Excess abdominal tissue (or an abdominal pannus) can occlude these venting solutions by completely submerging an open top or by covering the discrete vents on the upper layers of a device. This will lead to increased bag pressure and risk negative pressure injury.

Insofar as adhesive self-sticking is concerned in some commercially available devices, the position and flexibility of the adhesive layer for securing the device in place causes the material to crumple and stick to itself during placement. That can not only be annoying to the patient, but can lead to insecure placement on the patient, poor seal around the anatomy resulting in urine leakage, and thus may necessitate a replacement device. A need thus exist for a device configured for collecting urine from the penis of a patient which addresses the above shortcomings of the prior art. The subject invention does that.

### SUMMARY OF THE INVENTION

One aspect of this invention is a device configured for collecting urine from a penis of a patient. The device basically comprises a bag, a pad, an adhesive securement member, and a port. The bag is formed of a liquid impermeable flexible material and has an internal chamber and an entryway to the chamber. The entryway has a periphery. The chamber is configured for receipt of the penis of the patient though the entryway. The pad is formed of a flexible liquid wicking material configured for receipt of urine voided into the bag through the penis of the patient. The pad has a first portion disposed within the internal chamber, a second portion extending out of the internal chamber and in fluid communication with the ambient atmosphere, and an opening at the entryway. The first portion of the pad is contiguous with a first portion of the opening. The second portion of the pad is contiguous with a second portion of the opening. The adhesive securement member is secured to the bag and configured for adhesively mounting the device on the patient with the penis of the patient extending through the entryway into the internal chamber. The port is in fluid communication with the first portion of the pad. The port includes an external portion extending out of the bag and configured for connection to a source of controlled suction, whereupon when controlled suction from the controlled suction source is applied to the external portion of the port, urine voided into the internal chamber is carried through the first portion of the pad and out of the port by the controlled suction, with ambient air being drawn into the second portion of the pad to facilitate the carrying of the urine and drying of the pad.

In accordance with one preferred aspect of this invention the device comprises a spacer member formed of a wicking material and located outside of the bag. The spacer member is interposed between the adhesive securement member and the bag and is in fluid communication with the second portion of the pad. The spacer member is configured for enabling ambient air to be drawn therethrough and into the second portion of said pad.

In accordance with another preferred aspect of this invention the spacer member comprises a first layer of a flexible flange that surrounds the the entryway, and wherein the adhesive securement member comprises a second layer of the flexible flange.

In accordance with another preferred aspect of this invention the first and second layers are flat ring-shaped members secured to the bag at the periphery of the entryway.

In accordance with another preferred aspect of this invention the second layer comprises an adhesive on a flexible base layer, and wherein the device additionally comprises a removable liner releasably secured to the adhesive to protect the adhesive until the device is ready for use on the patient. The removable liner includes a projecting tab to facilitate removal of the liner from the adhesive.

In accordance with another preferred aspect of this invention the bag includes a front wall and a rear wall, and wherein the adhesive securement member is secured to the rear wall.

In accordance with another preferred aspect of this invention the flexible base layer is formed of spunlace polyester.

In accordance with another preferred aspect of this invention the adhesive is a silicone adhesive.

In accordance with another preferred aspect of this invention the bag includes a front wall and a rear wall, and wherein the adhesive securement member is secured to the rear wall, and the port is secured to the rear wall.

In accordance with another preferred aspect of this invention the bag and the pad are each of a generally tear-drop shape having a distal portion and a proximal portion, and wherein the entryway is located in the proximal portion and the port is located in the distal portion.

In accordance with another preferred aspect of this invention the bag includes a front wall and a rear wall, wherein the adhesive securement member is secured to the rear wall, wherein the port is secured to the rear wall, and wherein the device is configured so that when in use the rear wall faces the patient and the front wall faces away from the patient.

In accordance with another preferred aspect of this invention the front wall is transparent or translucent, whereupon the penis of the patient can be visualized through the front wall.

In accordance with another preferred aspect of this invention the pad is formed of polyester felt.

In accordance with another preferred aspect of this invention the pad and the spacer member are each formed of polyester felt.

Another aspect of this invention is a method of collecting urine from a penis of a patient. The method comprises providing a device for collecting urine from the penis of the patient. The device comprises a liquid impermeable flexible material bag having an internal chamber, an entryway to the internal chamber, an external port having a passageway in fluid communication with the internal chamber, and a pad of wicking material having a first portion located inside the internal chamber, and a second portion located outside the internal chamber and in fluid communication with the ambient atmosphere. The penis of the patient is inserted through the entryway, whereupon the penis is located within the internal chamber above the first portion of the pad. Controlled suction is applied to the external port and from there to the first portion of the pad, whereupon urine voided through the penis is carried by suction through the first portion of the pad and from there through the port for external collection, and wherein ambient air is drawn into the internal chamber through the second portion of the pad to facilitate movement of urine through the first portion of the pad and to cause drying of the first portion of the pad.

In accordance with one preferred aspect of the method of this invention the method additionally comprises adhesively mounting the device onto portions of the patient adjacent the penis.

In accordance with another preferred aspect of the method of this invention the portions of the patient adjacent the penis comprises portions of at least one leg of the patient.

In accordance with another preferred aspect of the method of this invention the bag includes a front wall and a rear wall. The front wall is transparent or translucent, and wherein the method comprises adhesively mounting the device on the patient with the rear wall facing the patient and the front wall facing away from the patient, whereupon the penis can be visualized through the front wall.

In accordance with another preferred aspect of the method of this invention the device comprises an adhesive securement member surrounding the entryway for adhesively mounting the device on the patient.

In accordance with another preferred aspect of the method of this invention the device additionally comprises a spacer member formed of a wicking material interposed between the bag and the adhesive securement member whereupon the spacer member is in fluid communication with the ambient atmosphere enabling ambient air to be drawn therethrough and into the second portion of the pad.

In accordance with another preferred aspect of the method of this invention the pad and the spacer member are each formed of polyester felt.

### DESCRIPTION OF THE DRAWING

FIG. 1 is a side elevation view of one exemplary embodiment of a male urinary collection device constructed in accordance with this invention and shown secured in an operative position on a patient with patient's penis being located within an internal chamber in a bag of the device;

FIG. 2 is an isometric view of the male urinary collection device of FIG. 1 shown taken from a slight oblique angle to show front-facing features of the device's components;

FIG. 3 is an isometric view of the male urinary collection device of FIG. 1 shown taken from a slight oblique angle to show rear-facing features of the device's components;

FIG. 4 is a reduced size exploded isometric view of the components of the device shown in FIGS. 1-3;

FIG. 5 is a reduced size top plan view of one of the components of the device of FIG. 1, namely, the front panel or wall forming one portion of the bag of the device;

FIG. 6 is a reduced top plan view of another of the components of the device of FIG. 1, namely, the wicking pad;

FIG. 7 is a reduced top plan view of another of the components of the device of FIG. 1, namely, the rear panel or wall forming another portion of the bag of the device;

FIG. 8 is a reduced top plan view of another of the components of the device of FIG. 1, namely, a padded spacer;

FIG. 9 is a reduced top plan view of another of the components of the device of FIG. 1, namely, an adhesive patch;

FIG. 10 is a reduced top plan view of another of the components of the device of FIG. 1, namely, a cover or liner sheet;

FIG. 11 is a reduced top plan view of another of the components of the device of FIG. 1, namely, suction coupling or port;

FIG. 12 is an enlarged isometric view of the wicking pad and the rear wall of a bag when assembled together;

FIG. 13 is an enlarged size longitudinal sectional view taken along line 13-13 of FIG. 12; and

FIG. 14 is a longitudinal sectional view similar to FIG. 13, but showing the suction port attached to the rear wall of the bag and showing a flange portion of the device secured to and below the rear wall, with the flange portion composed of the padding spacer, the adhesive patch, and the cover or liner.

### DETAILED DESCRIPTION OF THE PREFERRED EMBODIMENT

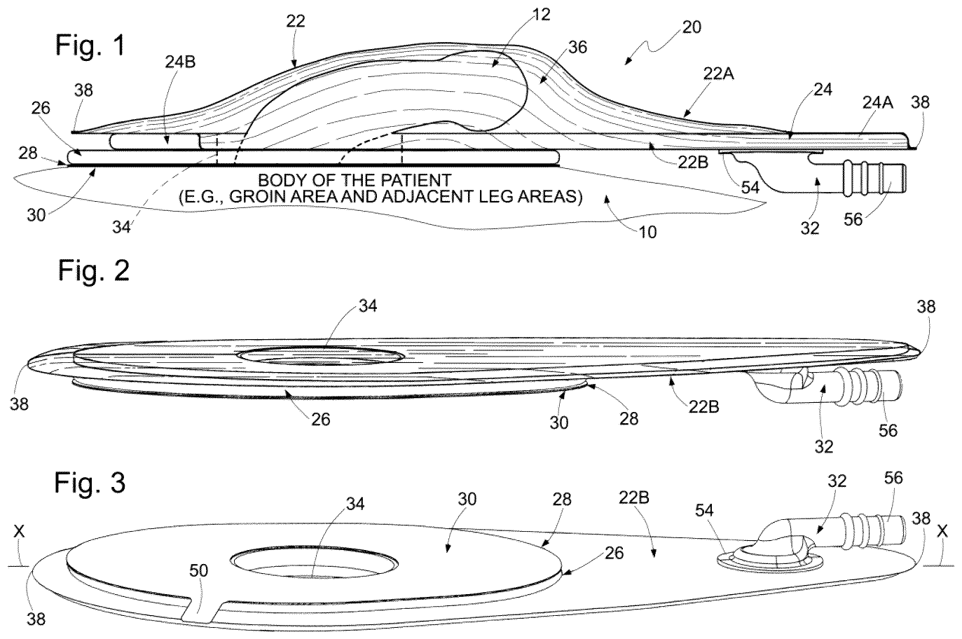

Referring now to the various figures of the drawing wherein like reference characters refer to like parts, there is shown at 20 in FIG. 1 one exemplary embodiment of a urinary collection device constructed in accordance with one exemplary preferred embodiment of this invention for collecting urine from the penis of a patient without making use of an indwelling catheter. That device and any other device constructed in accordance with this invention is particularly designed for use with a single patient over a prolonged period of time to transport urine out of the device for collection, and after use with that patient, the device is removed and disposed.

Before discussing the details of the embodiment 20 it must be understood that while that specific exemplary embodiment of the invention will be described more fully with reference to the accompanying drawings. Various other embodiments are contemplated within the scope of this invention. Thus, mention of other potential exemplary embodiments may also be provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiment may be embodied in many different forms and that neither should be construed to limit the scope of the disclosure. In any exemplary embodiments, well-known processes, well-known device structures, and well-known technologies are not described in detail.

The terminology used herein is for the purpose of describing the particular exemplary embodiment(s) only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath” “below” “lower” “above” “upper” “front” “rear” “top” “bottom” and the like may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

Turning back to FIG. 1 as mentioned above it shows one exemplary device 20 of this invention when in use. The device 20 basically comprises a flexible impermeable bag 22, a wicking insert or pad 24, a spacer 26, an adhesive securement member or patch 28, a cover or liner sheet 30, and a vacuum or suction connection port 32. The device 20 is configured to be adhesively secured to the body 10 of the patient at the patient's groin and adjacent leg areas by the adhesive patch 28, with the patient's penis 12 extending through an entryway 34 into an internal chamber 36 within the bag. The suction port 32 is configured to be connected to a source (not shown) of regulated suction to apply regulated suction at a desired operating level to the internal chamber in the bag. The wicking pad 24 includes a first (internal) portion 24A located within the interior chamber 36 of the bag and a second (external) portion 24B located outside of the bag. The internal and external portions of the wicking pad are in fluid communication with each other, e.g., they form respective portions of a unitary pad.

When the device 20 is in use the external portion 24B of the wicking pad is located interposed between the bag 22 and the body 10 of the patient, but in fluid communication with the ambient atmosphere. As such urine voided by the patient through the patient's penis 12 into the internal chamber 36 is transported through the internal portion 24A of the wicking pad by the combination of regulated suction applied to the suction port 32 and capillary action of the material making up the wicking pad, whereupon the urine flows out of the suction port and from there to an externally located urine collector, e.g., a receptable, bag or canister (not shown). Inasmuch as the external portion of the wicking pad is in fluid communication with the ambient atmosphere, air will be drawn from the ambient atmosphere through the external portion 22B to the internal portion 22A to facilitate the flow of urine to to suction port 32 and to effect the drying of the internal portion 22A.

The suction port 32 is configured to be connected to a section of conventional flexible tubing (not shown) interposed between the urine collector and the source of regulated suction. The source of regulated suction may be any conventional suction regulator, such as the Care4® regulator available from Boehringer Laboratories. In use with the subject invention the Boehringer suction regulator or any other suction regulator is connected to the conventional vacuum port in a hospital room or other medical care facility to adjust the level of line suction (e.g., ˜600 mmHg) at the vacuum port to a desired operating level, e.g., 125 mmHg.

The impermeable bag 22 is formed of a flexible material which is impermeable to fluids. In accordance, with one exemplary embodiment of the invention the bag comprises a front wall or panel 22A and a correspondingly shaped rear wall or panel 22B. The front wall or panel 22A may also be referred to as the top wall or panel. So too, the rear wall or panel may also be referred to as the bottom wall or panel. In any case each wall or panel is formed of a thin film that is flexible and transparent (or at least translucent). One particularly suitable film is polyurethane. The front panel 22A is positioned so that it faces away from the patient 10 when the device 20 is in place on the patient for use like shown in FIG. 1. At that time the rear wall or panel 22B faces toward the patient. The two panels 22A and 22B are connected together, e.g., heat sealed or welded, along their peripheries 38 to form the hollow internal chamber 36 between them. In the exemplary embodiments shown each panel 22A and 22B is of a generally tear-drop shape with a wide proximal end portion and a tapering distal portion. The proximal edge of the bag is rounded, as is the distal edge.

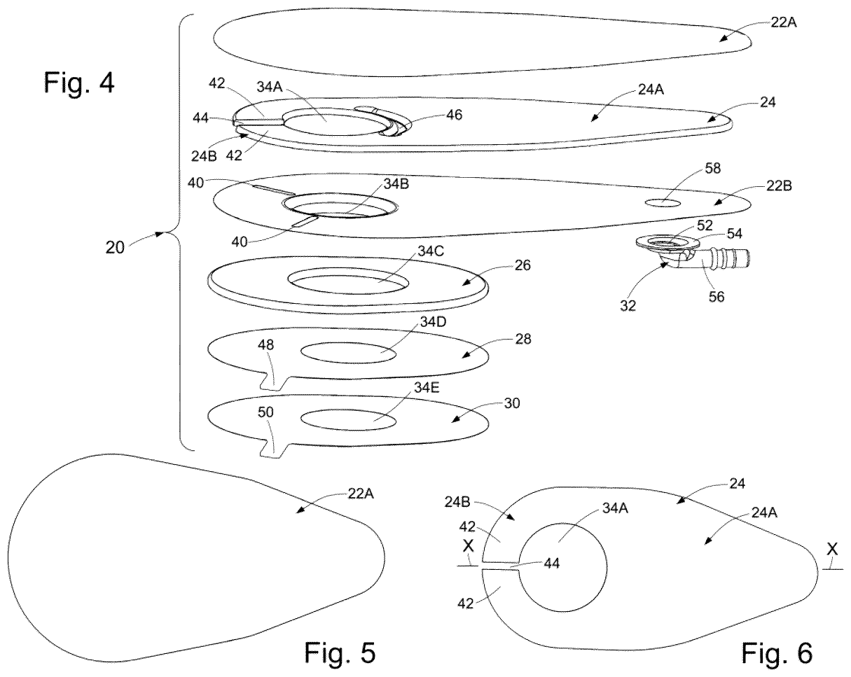

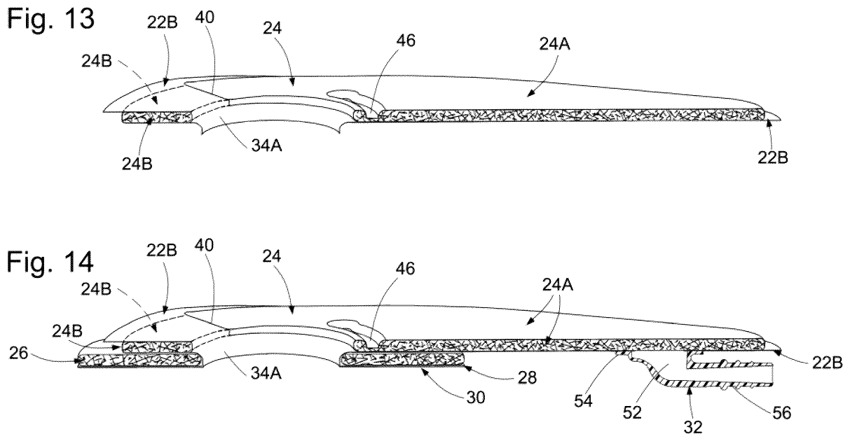

As mentioned earlier the bag 22 includes the entryway 34 for the patient's penis to extend therethrough so that it is in the position like shown in FIG. 1. In that position the head of the patient's penis is disposed over the internal portion 24A of the wicking pad for receiving urine voided therefrom. That entryway 34 is located in a flexible flange portion of the device 20 that is located and secured on the underside (the rear panel 22B) of the bag. As mentioned earlier the flange portion is a flexible construct composed of the spacer 26, the adhesive patch 28 and the cover or liner sheet 30. The entryway 34 extends through the flange portion of the device and is made up of an opening 34B in the rear wall 22B of the bag, and openings in the wicking pad 24, the spacer 26, the adhesive securement patch 28, and the liner sheet 30. In particular, as best seen in FIG. 4 the entryway 34 to the internal chamber 36 is made up of an opening 34A in the wicking pad 24, the opening 34B in the rear wall 22B of the bag, an opening 34C in the spacer 26, an opening 34D in the adhesive securement member or patch 28, and an opening 34E in the liner sheet 30. The opening 34B in the rear wall 22B of the bag is in the form of an annular flange as best seen in FIGS. 13 and 14. All of the openings 34A-34E are aligned with one another so that when the device 20 is assembled they collectively form the entryway 34 for the patient's penis into the internal chamber 36.

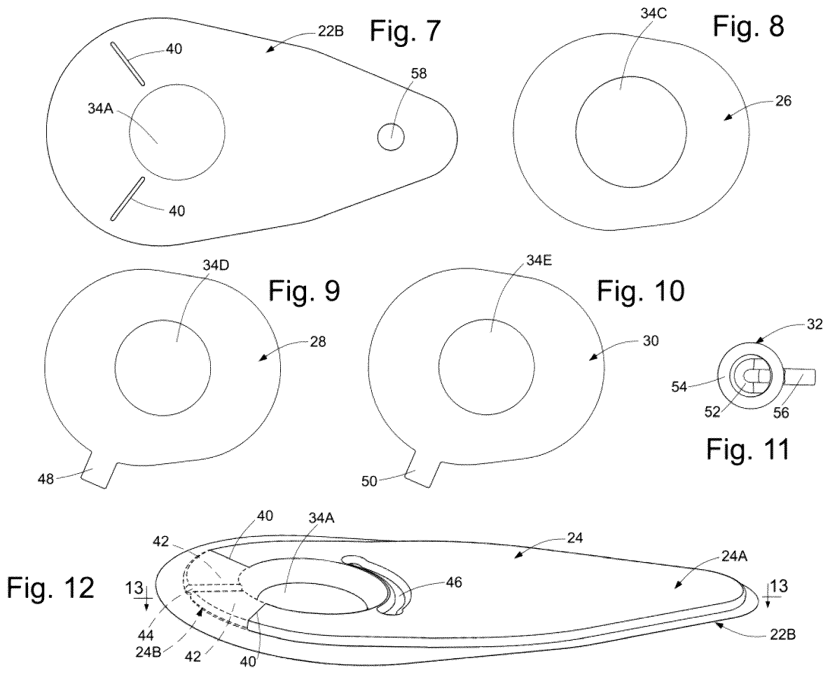

Turning now to FIGS. 4, 6, and 12-14 the details of the wicking pad 24 will now be described. As mentioned earlier the wicking pad is preferably a unitary member having an internal portion 24A and an external portion 24B which are in fluid communication with each other. The wicking pad is also of generally tear-drop shape like that of the bag 22 and sized just slightly smaller than the size of the bag so that the wicking pad fits within the confines of the periphery of the bag. In particular, the internal portion 24A of the wicking pad fits within the distal portion of the internal chamber 36 in the bag, with the external portion 24B extending through elongated slots 40 in the rear panel 22B, whereupon the external portion extends outside of the bag and underlying the rear panel at the proximal portion of the bag. As best seen in FIG. 7 the slots 40 extend generally radially from respective points adjacent the proximal side of the opening 34A to accommodate respective wings 42 making up the external portion 24B of the wicking pad. As best seen in FIG. 6 the wings 42 are defined by the proximally located portion of the pad contiguous with the proximally located portion of the opening 34A and a slot 44 which extends along the longitudinal central axis X of the device 20 from the proximal end of the wicking pad to the opening 34A. Each of the wings 42 is configured to be extended from within the interior chamber 36 through the heretofore identified slots 40 in the rear panel 22B of the bag, whereupon each wing is disposed under the rear panel of the bag and in fluid communication with the ambient atmosphere as clearly seen in FIGS. 1 and 14. The wicking pad is fixedly secured to the rear panel 22B of the bag 22, by an arcuate heat seal line 46 located immediately adjacent the distal side of the opening 34A. The formation of the heat seal 46 compresses the polyester felt material of the pad 24 to result in a recess within the front side of the pad as can be seen in FIGS. 4, and 12-14.

The wicking pad 24 is provided to achieve efficient transport or flow of urine voided into the bag to the port 32. In fact, the wicking pad acts as both a highway for liquid (urine) movement and a venting pathway to pull in air from the external environment (ambient atmosphere). Since the wicking pad is the sole component within the bag where the urine is voided, the key to its efficiency in transporting that urine is the material making up the pad. One particularly good material constitutes homogenous polyester felt, although some other suitable wicking materials may be used. As should be appreciated by those skilled in the art felt excels at wicking liquid.

When the device 20 is in use and connected to a source of suction and a collection device, urine voided into the interior chamber 36 through the patient's penis (which overlies the internal portion 24A of the pad) contacts the felt material and is immediately drawn into the felt material. The urine then spreads out passively across an area (e.g., approximately 1.5 inch in radius for the size wicking pad described above). The suction applied to the interior chamber through the suction port pulls urine from the pad in the area directly adjacent to the port and creates a region of lower liquid concentration. Urine accumulated in the surrounding regions rapidly wicks to the drier region where it is continuously siphoned through the port and out of the device 20. The removal of urine from the device proceeds until the wicking pad is dry.

Due to the wicking properties of the felt material of the pad, it doesn't “hold” onto the urine like a more absorptive material would. This property not only allows the urine to easily enter and exit, but also to rapidly move through the pad as long as there is a moisture gradient. The combination of passive wicking and suction induced moisture gradient allows for the rapid expulsion of urine and maximizes the drying of the device and the patient.

In accordance with one exemplary embodiment of the device of this invention the polyester felt wicking pad 24 is approximately 0.1875 inch thick. The film making up the polyurethane panels 22A and 22B of the bag is approximately 0.003″ thick. In accordance with one exemplary embodiment of the device of this invention the length of the bag is approximately 11 inches and its maximum width is approximately 6 inches. It should be noted that the thickness dimensions of the bag's walls and the wicking pad are merely exemplary. Thus either may differ depending on the particular materials chosen for the wicking pad, the desired wicking capability of the pad, and overall flexibility of the device 20.

Turning now to FIGS. 1-3, 9 and 14 the details of the adhesive securement member or patch 28 will now be described. That member serves to adhesively secure the device onto the body of the patient to hold it in place comfortably and safely. The adhesive securement patch basically comprises a ring-shaped member formed of a base layer of a flexible material, e.g., spunlace polyester. A tab 48 projects outward from the base layer at a portion of the periphery of the base layer. The undersurface of the base layer is coated with a silicone adhesive, except for the undersurface of the tab 48, which is uncoated. The patch 28 is fixedly secured, e.g., heat sealed, to the rear panel 22B by a circular heat seal in the base layer contiguous with the opening 34 and extending about the entire periphery of that opening.

In order to protect the silicone adhesive until the device 20 is ready for releasable securement mounting on the patient, the heretofore mentioned cover or liner sheet 30 is provided. That liner sheet is of the same size and shape as the patch 28 and is formed of any suitable film material, e.g., polypropylene film such as available from 3M under model designation 2476P. The liner sheet includes a projecting tab 50 which is of the same size and shape as the tab 48 and overlies the tab 48. Since the tab 48 of the adhesive patch does not include an adhesive thereon, the overlying tab 50 of the liner sheet can be readily grasped by a user to peel the liner sheet off of the adhesive patch thereby exposing the adhesive.

With the adhesive coating of the adhesive patch exposed, the device 20 can be juxtaposed with respect to the patient so that the penis of the patient can be extended through the aligned openings whereupon the head of the penis is located within the bag 22 and the position of the penis therein visualized through the transparent or translucent front wall 22A of the bag. The adhesive securement patch 28 can then be pressed down so that its exposed adhesive engages the body of the patient around the base of the penis, e.g., the patient's pubic area and contiguous portions of the patient's legs, thereby releasably mounting the device on the patient.

Turning now to FIGS. 1-3, 8 and 14 the details of the spacer 26 will now be described. The spacer constitutes an external component of the device 20 inasmuch as it is located outside of the bag 22. The spacer is in the form of a pad composed of the same material as the wicking pad 24 (or any material that allows for a substantial passage of air to adequately vent the internal chamber 36 pressure of the bag 22). It is also of the same thickness as the wicking pad (but could be thicker or thinner, if desired), and is of the same shape and coextensive size as the securement patch 28. The spacer does not include any tab projecting from its periphery and its opening 34C is slightly larger than the openings in the other components which make up the entryway 34. For example the diameter of the opening 34C of the spacer is approximately 2.4 inches, whereas the diameter of the opening of 34A is approximately 2.5 inches, the diameter of the opening of 34B is approximately 1.9 inches, and the diameters of openings 34D and 34E are approximately 2 inches. The slightly enlarged size opening 34C in the spacer is provided to prevent crumpling of the materials making up the other layers of the devices's components that its larger size encompasses.

The spacer 26 provides two functions. First, it facilitates placement of the adhesive on the patient when securing the device. Second, it acts as the external component of the venting pathway. Regarding the first function, as discussed above thin adhesive backed fabrics, like that of some prior art devices, can be unwieldy during normal use. However, if provided with a stiff backing they are much less likely to crumple. The spacer 26 accomplishes that action by acting as a layer of relatively stiff material to hold the adhesive fabric against the patient during placement on a patient.

It should be noted that the spacer is not adhered to the bag itself. Rather, the spacer is held in place interposed between the bag 22 and the adhesive patch 28 by the heat seal between rear panel 22B of the bag (which extends along the entire periphery of the opening 34B) and the base layer of the adhesive patch 28 (which extends along the entire periphery of the opening 34D). As such, the spacer can move with respect to the bag and the adhesive patch, while acting as a platform to support and press the adhesive in place.

With respect to the second function of the external spacer, i.e., its role in the venting pathway of the device, it cooperates with the externally located portion 24B of the wicking pad. In this regard, the front or upper surface of the spacer is located immediately below and abuts the undersurface or bottom of the wings 42 making up the external portion of the wicking pad. Being formed of the same wicking material as the wicking pad ambient air is enabled to flow into the spacer and from there into the abutting wings 42, where that air joins with the air entering the wings themselves from other directions, e.g., the edges of the wings. The air entering the wings of the wicking pad flows into the contiguous internal portion 22A of the wicking pad which facilitates the transport of the urine through the wicking pad to the port 32, while also assisting in drying of the wicking pad, as mentioned earlier. The impervious material of the rear panel 22B contiguous with the slots 40 closely engage the contiguous portions of the wings 42 extending therethrough to preclude any urine leaking out of the interface of those slots and the wings.

As also mentioned earlier, the internal pressure of the device must be controlled to prevent patient injury due to excessive negative pressure. This can be partially controlled by proper setting of the regulated suction level provided to the port 32. If, however, the device 20 is not provided with the desired level of regulated suction (i.e., suction at the specified source setpoint), such as could occur if unregulated wall/line suction is directly applied to the port 32 of the device 20, excessive negative pressure will not result due to the action of the spacer 26. In this regard, the spacer acts as a large air intake providing sufficient air flow into the device to prevent pressure buildup even at line pressures (˜600 mmHg). Since the spacer 26 abuts and is in fluid communication with the wings 42 it completes the airflow pathway into the bag 22. Once inside the chamber 36, the air will flow over the patient's anatomy and out through the suction port with any liquid.

As should be appreciated by those skilled in the art the construction of the device 20 (or any other device constructed in accordance with this invention) prevents the problems of improper venting of some prior art devices, wherein the top side vents or open ends of such prior art devices can be easily occluded by patients with large abdomens. That action is achieved by the the subject invention positioning the air intake on the underside or bottom of the device. The benefit of that placement is dictated by anatomy: the bottom of the device interfaces with portions of the patient's legs while the top vents/open ends of prior art devices must contend with the smooth, uninterrupted skin of an abdominal pannus. Legs, conversely, are separate entities that curve away centrally from the groin area; paired with the large air intake discussed above, even the largest patients should not be able to fully occlude the entire exposed circumference of the external spacer 26 of the subject invention when the device is in place in use.

Turning now to FIGS. 4, 11 and 14, it can be seen that the suction coupling or port 32 basically comprises a tubular L-shaped member having a central passageway 52 extending the entire length of the member from one end to the other end. The port can be formed of any conventional polymer such as used to make flexible medical tube fittings. One particularly suitable polymer is polyurethane/polyvinylchloride that is somewhat flexible and of a suitable durometer, e.g., 70-90 Shore A. The inner end of the port 32 is in the form of a circular flange 54 which is fixedly secured to the bag 22, as will be described later. The outer end 56 of the port is configured to be received within the open end of flexible medical tubing.

As best seen in FIG. 7 the rear panel 22B includes a small diameter, e.g., 0.70 inch, aperture 58 located closely adjacent the distal end of the panel. The flange 54 of the port 32 is fixedly secured, e.g., welded, to the portion of the rear panel 22B contiguous with the aperture 58, whereupon the aperture 58 is in fluid communication with the passageway 52 in the port. Accordingly, the interior of the chamber 36, and the internal portion 24A of the wicking pad will be in fluid communication with the passageway 52 in the port 32 via the aperture 58.

In order to connect the device 20 to the source of regulated suction and the collector or receptacle for collecting the urine withdrawn from the device a section of conventional tubing (not shown) formed of any suitable flexible material, e.g., flexible PVC tubing, like used in hospitals to carry fluids via suction is provided. To that end, the portion 56 of the port 32 is inserted within the proximal end of the tubing section and the distal end of the tubing section is connected to any suitable urine collector, e.g., a bag, cannister, etc. (not shown). Since the portion 56 of the port 32 extends generally parallel to the rear panel 22B when coupled to a section of flexible tubing as just described it and the tubing will extend parallel to the legs of the patient in the interest of patient comfort.

The urine collector (not shown) is connected to the output of the suction regulator (not shown) by another section of flexible tubing (not shown). That other section of flexible tubing is connected to the line suction port (not shown) of the hospital or other health care facility where the device 20 will be used. Thus, once the device 20 is secured in place on the patient, regulated suction at the desired level, e.g., 125 mmHg, will be provided to the internal chamber via the port 32. Confirmation of patient anatomy within the device 20 can be performed by direct visualization through the front wall 22A of the bag whenever necessary. When a replacement of the device 20 is required, the adhesive can be safely removed from the patient before disposal of the device per hospital protocol.

As should be appreciated from the above description the exemplary device 20, and any other device constructed in accordance with this invention and its method of use offers various clinical advantages. They are: (1) minimal gravity dependence due to high-capacity passive wicking; (2) large, venting pathway on the rear or bottom side of the device prevents occlusion and resulting pressure injury; (3) wicking ability combined with uninterrupted airflow act to keep sensitive anatomy dry, preventing skin breakdown or other damage; (4) self-sticking of adhesive during placement is limited due to external spacer backing; (5) conforming top layer facilitates initial placement of the patient's penis within the bag; and (6) the device use fits into current workflow for hospital staff, no unique/advanced training required, such staff can readily understand use of the device based on their familiarity with existing male urine collection devices).

Without further elaboration the foregoing will so fully illustrate our invention that others may, by applying current or future knowledge, adopt the same for use under various conditions of service.

We claim:

1. A device for collecting urine from a penis of a patient comprising:
a bag formed of a liquid impermeable flexible material, said bag having an internal chamber and an entryway to said internal chamber, said entryway having a periphery, said internal chamber being configured for receipt of the penis of the patient though said entryway;
a pad formed of a flexible liquid wicking material configured for receipt of urine voided into said bag through the penis of the patient, said pad having a first portion disposed within said internal chamber, a second portion extending out of said internal chamber and in fluid communicatin with the ambient atmosphere, and an opening at said entryway, said first portion of said pad being contiguous with a first portion of said opening, said second portion of said pad being contiguous with a second portion of said opening;
an adhesive securement member secured to said bag and configured for adhesively mounting said device on the patient with the penis of the patient extending through said entryway into said internal chamber; and
a port in fluid communication with said first portion of said pad, said port including an external portion extending out of said bag and configured for connection to a source of controlled suction, whereupon when controlled suction from said controlled suction source is applied to said external portion of said port urine voided into said internal chamber is carried through said first portion of said pad and out of said port by said controlled suction, with ambient air being drawn into said second portion of said pad to facilitate the carrying of the urine through said first portion of said pad and drying of said first portion of said pad.

2. The device of claim 1 additionally comprising a spacer member formed of a wicking material and located outside of said bag, said spacer member being interposed between said adhesive securement member and said bag and being in fluid communication with said second portion of said pad, said spacer member being configured for enabling ambient air to be drawn therethrough and into said second portion of said pad.

3. The device of claim 2 wherein said spacer comprises a first layer of a flexible flange that surrounds said entryway, and wherein said adhesive securement member comprises a second layer of said flexible flange.

4. The device of claim 3, wherein said first and second layers are flat ring-shaped members secured to said bag at said periphery of said entryway.

5. The device of claim 4 wherein said second layer comprises an adhesive on a flexible base layer, and wherein said device additionally comprises a removable liner releasably secured to adhesive to protect said adhesive until said device is ready for use on the patient, said removable liner including a projecting tab to facilitate removal of said liner from said adhesive.

6. The device of claim 5 wherein said flexible base layer is formed of spunlace polyester.

7. The device of claim 6 wherein said adhesive is a silicone adhesive.

8. The device of claim 1 wherein said bag includes a front wall and a rear wall, and wherein said adhesive securement member is secured to said rear wall, and said port is secured to said rear wall.

9. The device of claim 1 wherein said bag and said pad are each of a generally tear-drop shape having a distal portion and a proximal portion, and wherein said entryway is located in said proximal portion and said port is located in said distal portion.

10. The device of claim 1 wherein said bag includes a front wall and a rear wall, wherein said adhesive securement member is secured to said rear wall, wherein said port is secured to said rear wall, and wherein said device is configured so that when in use said rear wall faces the patient and said front wall faces away from the patient.

11. The device of claim 10 wherein said front wall is transparent or translucent, whereupon the penis of the patient can be visualized through said front wall.

12. The device of claim 1 wherein said pad is formed of polyester felt.

13. The device of claim 2 wherein said pad and said spacer member are each formed of polyester felt.

14. A method of collecting urine from a penis of a patient comprising:
providing a device for collecting urine from said penis of said patient, said device comprising a liquid impermeable flexible material bag having an internal chamber, an entryway to said internal chamber, an external port having a passageway in fluid communication with said internal chamber, and a pad of wicking material having a first portion located inside said internal chamber, and a second portion located outside said internal chamber and in fluid communication with the ambient atmosphere;
inserting said penis of said patient through said entryway, whereupon said penis is located within said internal chamber above said first portion of said pad; and
applying controlled suction to said external port and from there to said first portion of said pad, whereupon urine voided through said penis is carried by suction through said first portion of said pad and from there through said port for external collection, and wherein ambient air is drawn into said internal chamber through said second portion of said pad to facilitate movement of urine through said first portion of said pad and to cause drying of said first portion of said pad.

15. The method of claim 14 wherein said method additionally comprises adhesively mounting said device onto portions of said patient adjacent said penis.

16. The method of claim 15, wherein said portions of said patient adjacent said penis comprise portions of at least one leg of said patient.

17. The method of claim 14 wherein said bag includes a front wall and a rear wall, said front wall being transparent or translucent, and wherein said method comprises adhesively mounting said device on said patient with said rear wall facing said patient and said front wall facing away from said patient, whereupon said penis can be visualized through said front wall.

18. The method of claim 15 wherein said device comprises an adhesive securement member surrounding said entryway for adhesively mounting said device on said patient.

19. The method of claim 18 wherein said device additionally comprises a spacer member formed of a wicking material interposed between said bag and said adhesive securement member whereupon said spacer member is in fluid communication with said ambient atmosphere enabling ambient air to be drawn therethrough and into said second portion of said pad.

20. The method of claim 19 wherein said pad and said spacer member are each formed of polyester felt.
